(19) United States
(12) Patent Application Publication
(10) Pub. No.: US 2003/0224346 A1
(43) Pub. Date: Dec. 4, 2003

(54) Chromatographic method

(76) Inventor: Goran Karlsson, Ekero (SE)

Correspondence Address: FISH & RICHARDSON PC, 225 FRANKLIN ST, BOSTON, MA, 02110, US

(21) Appl. No.: 10/371,548

(22) Filed: Feb. 21, 2003

(30) Foreign Application Priority Data
Feb. 21, 2002 (SE) 0200507-2

(51) Int. Cl. C12Q 1/00

(52) U.S. Cl. 435/4

(57) ABSTRACT

Methods for determining the molecular weight distribution in a sample of an anionic polysaccharide is provided. The methods generally include: providing a sample of an anionic polysaccharide with an average molecular weight in the range of from 0.05 to 10 MDa; applying the sample to an anion-exchange chromatography column so as to immobilize the polysaccharide to the column; eluting the immobilized polysaccharide while recording a chromatogram of the amount of polysaccharide eluted as a function of time; and determining the molecular weight distribution in the polysaccharide sample through analysis of the chromatogram. Also provided is use of anion-exchange chromatography for the determination of molecular weight distribution in a sample of an anionic polysaccharide with an average molecular weight in the range of from 0.05 to 10 MDa.

### CROSS REFERENCE TO RELATED APPLICATIONS

This application claims priority from Swedish Patent Application No. 0200507-2, filed Feb. 21, 2002. The entire content of this prior application is incorporated herein by reference in its entirety.

### TECHNICAL FIELD

The present invention relates to the determination of molecular weight distribution in samples of anionic polysaccharides. More particularly, the invention is concerned with a chromatographic method for such determination.

### TECHNICAL BACKGROUND

The molecular weight of a polysaccharide is one of its most fundamental characterizing features, and one that has a profound impact on the function and usefulness of the polysaccharide in various applications. However, it is a parameter that has proven very difficult to measure. The difficulties arise due to a number of polysaccharide properties, such as their polydispersity, non-ideal thermodynamics, conformational flexibility and self-association at high concentrations. The difficulties encountered in attempts at determining the molecular weight of polysaccharides are even more pronounced when the goal is to determine the molecular weight distribution in a polysaccharide sample. These difficulties are problematic, since the knowledge of molecular weight and molecular weight distribution of polysaccharides is of crucial importance when it comes to elucidating their role in biochemistry and their possible biotechnological, medical and other commercial applications. A general review of the topic of determination of the molecular weight of polysaccharides is given in SE Harding et al, Advances in Carbohydrate Analysis 1:63-144 (1991).

In the food industry, low molecular weight species present in too great an amount in polysaccharide gelling or thickening agents diminishes the performance of such agents, and there have also been reports of possible toxicity of low molecular weight forms in other food additives. Polysaccharides are also used in the oil industry for a variety of purposes, e.g., for mobility control, and their effectiveness in fulfilling these purposes is largely determined by their molecular weight. There is an increasing use of polysaccharides in drug delivery systems within the pharmaceutical industry, where, again, the performance of the polysaccharides used is dependent on their molecular weight properties. This is so for example with regard to properties related to transport through the polysaccharide of active substances, e.g., in dosage forms for controlled release. Polysaccharides (such as dextrans, schizophyllan, hyaluronic acid, heparin, chondroitin sulfate, and chitosan) are also applied or injected to patients for use as therapeutics themselves. In addition to specific physiological properties depending on the chemical structure of the polysaccharide, such agents must also frequently comply with requirements as to physical properties, such as viscosity, osmotic pressure, and gelling, which properties are greatly dependent on the molecular weight and molecular weight distribution.

An illustrative example of a medically important, anionic polysaccharide is hyaluronic acid. Hyaluronic acid (HA) was first isolated from the vitreous body of the eye (K Meyer and J W Palmer, J Biol Chem 107:629-634 (1934)), and has been shown to be a glucosaminoglucan of the type (-GlcNAc-GlcUA-)ₙ where GlcNAc is N-acetyl-D-glucosamine and GlcUA is D-glucuronic acid. This polymer has a helical conformation, and is found in such tissues as the vitreous body of the eye, the cartilage, and the synovial fluid of the joints (TC Laurent and RE Fraser, FASEB J 6:2397-2404 (1992)). The molecular weight of the hyaluronic acid polymer is in the range of from 10 000 Da up to about 1×10⁷ Da (T Sugiyama et al J Appl Ther Res 2:141-145 (1998)). Commercial pharmaceutical products of HA have been used for treatment of joint diseases, e.g., rheumatoid arthritis, and have also been used in ophthalmic surgery. The average molecular weight of HA for these products lies within a range of about 0.5-5 MDa. Several reports have been published which reveal that low molecular weight HA (HA with a molecular weight of 0.5 MDa or less) is a strong inflammatory mediator in various tissues, and that this inflammatory response is evoked by chemokine gene expression (CM McKee et al, J Clin Invest 98:2403-2413 (1996)). Another example is results which show that low molecular weight HA can induce the production of IL-8 from cultured umbilical fibroblasts (N Kanayama et al, Pediatr Res 45:510-514 (1999)). IL-8 is generally considered as a strong neutrophilic chemotactic factor, which induces an inflammatory reaction in various tissues. Therefore, an important step in the process and analysis of these products is the determination of the average molecular weight and the molecular weight distribution of hyaluronic acid.

Several methods have been employed for the determination of molecular weight of polysaccharide samples containing species in the molecular weight range of 0.1 MDa and above. These include size-exclusion chromatography (SEC) (Sugiyama et al, supra), agarose gel electrophoresis (N Kanayama et al, supra), capillary electrophoresis (S Hayase et al, J Chromatogr A768:295-305 (1997)), viscosimetry (T Yanaki and T Yamaguchi, Biopolymers 30:415-425 (1990)), and laser light scattering (LALLS) detectors (C Kvam et al Anal Biochem 211:44-49 (1993)). Mass spectrometry can not be used for these high molecular weights (above 0.5 MDa). Furthermore, only a few of the methods outlined above result in more than an average value of the molecular weight.

Information about the distribution of molecular weights in a sample of a commercial polysaccharide product of high molecular weight can thus, in practice, only be obtained through light scattering techniques and size exclusion chromatography. Still, the use of any of these two methods is associated with problems. Methods employing light scattering are extremely sensitive to dust or macromolecular aggregates in the sample to be analyzed, since such contamination leads to serious errors. Size exclusion chromatography is the most popular and widely used technique for determination of molecular weight distribution of polysaccharides, but suffers from the drawback that the maximum molecular weight that can be analyzed with this method often is rather low. This means that the molecular weight distribution analysis of commercial polysaccharide samples, in which a significant proportion of the individual polymer molecules have a molecular weight above a certain maximum molecular weight, is not feasible. For example, there are important commercial hyaluronic acid products which have an average molecular weight of around 4-5 MDa, while size exclusion chromatography of hyaluronic acid may only be performed on molecular weights of up to about 3 MDa.

Y Zhang et al, Anal Biochem 250:245-251 (1997), describe an approach to separate oligosaccharides using high performance anion-exchange chromatography coupled to means for pulsed amperometric detection (HPAEC-PAD). In this report, oligo/poly-sialic acids with a degree of polymerization of up to 80 were separated, corresponding to molecular weights of about 24 kDa or less. Other workers have separated smaller oligosaccharides using the same approach (see e g K N Price et al, Carbohydrate Research 303:303-311 (1997)). The type of chromatography used in these experiments demands a special type of chromatography equipment and column, and can only be performed at pH values sufficiently high to create negatively charged OH⁻ groups on the oligosaccharides that are analyzed. In the report quoted above, Y Zhang et al operate with a pH value of about 13.

From the above it is clear that there is a need for new and improved techniques for analysis of molecular weight distribution of polysaccharides, which complement the existing techniques and offer practical and efficient alternatives.

### SUMMARY OF THE INVENTION

Thus, it is an object of the present invention to provide such alternatives for the determination of molecular weight distribution of polysaccharides.

It is another object of the present invention to enable the straightforward and simple analysis of polysaccharide samples of a high molecular weight, such as above the threshold molecular weight for size exclusion chromatography.

Yet another object of the invention is to provide a method which is easily adapted to existing standard technologies for chromatography and the analysis of chromatograms.

A further object of the present invention is to establish a new use for conventional anion-exchange chromatography.

These and other objects which will be apparent to the skilled man from the following detailed description, are met by the invention as claimed. Thus, the present invention provides, in one of its aspects, a method for determining the molecular weight distribution in a sample of an anionic polysaccharide, comprising the steps of:

(i) providing a sample of an anionic polysaccharide with an average molecular weight in the range of from 0.05 to 10 MDa;

(ii) applying the sample to an anion-exchange chromatography column so as to immobilize the polysaccharide to the column;

(iii) eluting the immobilized polysaccharide while recording a chromatogram of the amount of polysaccharide eluted as a function of time; and

(iv) determining the molecular weight distribution in the polysaccharide sample through analysis of the chromatogram obtained in step (iii).

In another aspect, the present invention provides use of anion-exchange chromatography for the determination of molecular weight distribution in a sample of an anionic polysaccharide with an average molecular weight in the range of from 0.05 to 10 MDa.

Thus, the present invention is based on the surprising finding that conventional anion-exchange chromatography may advantageously be used to determine the molecular weight distribution of a sample of an anionic polysaccharide. Among other advantages, the invention offers the possibility of extending upwards the range of molecular weights that may be analyzed by chromatography, in comparison with previously employed size exclusion methods and with the HPAEC-PAD method used to determine molecular weight distribution of oligosaccharides. The invention is not critically dependent on any particular anion-exchange chromatography system, but may for example be carried out using commercially available HPLC-equipment. Furthermore, the invention offers a practical and simple alternative to light scattering techniques, since it is not as critically dependent on samples without any dust or macromolecular aggregates.

### BRIEF DESCRIPTION OF THE DRAWINGS

FIGS. 1A-1E are graphs depicting anion-exchange chromatography of: (A) water blank; (B) hyaluronic acid, 0.1 MDa; (C) hyaluronic acid, 0.25 MDa; (D) hyaluronic acid, 0.5 MDa; and (E) hyaluronic acid, 1 MDa. Elution was by a gradient of 20-225 mM sodium sulfate.

FIGS. 2A-2E are graphs depicting anion-exchange chromatography of: (A) water blank; (B) hyaluronic acid, 1 MDa; (C) hyaluronic acid, 3 MDa; (D) hyaluronic acid, 4 MDa; and (E) hyaluronic acid, 5 MDa. Elution was by a gradient of 175-225 mM sodium sulfate.

FIGS. 3A-3B are standard curves with retention time plotted against molecular weight of hyaluronic acid. FIG. 3A depicts hyaluronic acid, 0.1-1 MDa, eluted by a gradient of 20-225 mM sodium sulfate. FIG. 3B depicts hyaluronic acid, 1-5 MDa, eluted by a gradient of 175-225 mM sodium sulfate. A third degree polynomial curve fit model was used as indicated.

FIGS. 4A-4B are graphs depicting hyaluronic acid standards analyzed as unknowns. By manual splitting of the integrated peaks at the retention times for respective molecular weight standard, the molecular weight distribution for the selected ranges was obtained, as indicated in the chromatograms. FIG. 4A depicts hyaluronic acid, 0.25 MDa, eluted by a gradient of 20-225 mM sodium sulfate. FIG. 4B depicts hyaluronic acid, 4 MDa, eluted by a gradient of 175-225 mM sodium sulfate.

### DETAILED DESCRIPTION OF THE INVENTION

The method of the invention uses as its starting point a sample of an anionic polysaccharide, which is provided in step (i). This sample may be a sample of a commercial product, the molecular weight distribution of which is to be studied for reasons of quality assurance, but may equally well be a sample which is to be studied and characterized in a context of basic research. The invention is not limited to any specific application or field of use. The anionic polysaccharide can be selected from the group consisting of hyaluronic acid, chondroitin sulfates, keratan sulfates, dermatan sulfates, heparin sulfate and heparan sulfate. A particularly preferred anionic polysaccharide is hyaluronic acid. The polysaccharide sample preferably has an average molecular weight of no less than 0.05 MDa, more preferably no less than 0.1 MDa. The method of the invention may be applied in the analysis of samples comprising very high molecular weights, such as a molecular weight of individual polymer chains in the sample of up to 10 MDa, or at any rate up to about 5 MDa. It is furthermore preferred that the sample to be analyzed has a high degree of purity. Especially contaminations of negatively charged species, such as certain proteins or sulfated polysaccharides, are preferably kept to a minimum. This is because such contaminants may bind strongly to the anion-exchange materials used, and thus disturb the analysis. In accordance with this, the sample preferably has a degree of purity such that the contamination of negatively charged species in the sample is less than 5%, preferably less than 1%, more preferably less than 0.1%.

In step (ii) of the method of the invention, the polysaccharide sample is applied to an anion-exchange chromatography column, under conditions that are such that the polysaccharide is immobilized to the column. The invention is not restricted to any specific anion-exchange material, but use is advantageously made of strong or weak anion-exchange chromatography columns that are commercially available, such as for example columns with functional groups selected from the group consisting of aminoethyl, diethylaminoethyl, dimetylaminoethyl, polyethyleneimine, trimethylaminomethyl, trimethylaminohydroxypropyl, diethyl-(2-hydroxypropyl)aminoethyl, quaternized polyethyleneimine, triethylaminoethyl, trimethylaminoethyl and 3-trimethylamino-2-hydroxypropyl. Among these, strong anion exchangers with functional groups comprising quaternary amine are preferred, e.g., trimethylaminomethyl, trimethylaminohydroxypropyl, diethyl-(2-hydroxypropyl)aminoethyl, quaternized polyethyleneimine, triethylaminoethyl, trimethylaminoethyl and 3-trimethylamino-2-hydroxypropyl. The conditions under which the polysaccharide is immobilized to the column may be easily ascertained by the skilled person without undue experimentation. However, it should be pointed out that the method according to the present invention does not depend on an elevated pH value in the mobile phase, but may advantageously be carried out using a mobile phase having a neutral or moderately basic pH. Thus, the pH value in the mobile phase can lie within the range from pH 4 to pH 11, e.g., within the range from pH 6 to pH 9. Use of a too high pH value, such as above pH about 12, may risk of alkaline hydrolysis of the polysaccharide at such an elevated pH.

Elution of the immobilized polysaccharide in step (iii) of the method of the invention is generally performed using a concentration gradient of a solution of an elution salt, in accordance with known chromatographic procedure. The elution salt used can, for example, be chosen among standard elution salts for ion-exchange chromatography, such as lithium chloride, sodium chloride, potassium chloride, magnesium chloride, calcium chloride, barium chloride, sodium acetate, lithium perchlorate, sodium sulfate, magnesium sulfate, potassium phosphate and potassium sulfate, or other elution salts known to the person of skill in the art. Depending on the detection method used, certain salts may be preferred over others, due to for example absorbance properties. Thus, for detection based on absorbance of ultraviolet light, sulfates may be preferred due to their low absorbance. More generally, sulfates are also advantageously used because of their relatively high ionic strength, which means that a lower salt concentration is needed for elution. A particularly preferred elution salt for use in the method of the invention is sodium sulfate.

During elution, a chromatogram is recorded, showing the amount of polysaccharide eluted as a function of time. Shorter polymer chains, e.g., chains having a lower molecular weight and a smaller net electrical charge, will be eluted at a lower elution salt concentration than longer polymer chains, and will thus be detected earlier during elution. Thus, the relative elution time of an individual polysaccharide chain in the sample will correspond to the relative size of that polysaccharide chain. The detection of eluted polysaccharide may be performed using any known detecting means, suitably positioned in connection with the outlet of the chromatography column. Such detectors are well known to the person of skill in the field, and certain detectors may be preferred over others due to reasons of economy, ease of access or other practical considerations.

The analysis in step (iv) of the chromatogram obtained in step (iii) has for an object to provide the desired information on the molecular weight distribution of the polysaccharide sample. This information may for example be expressed as a certain percentage of the polymer chains in the sample having a weight below a first value, another percentage having a weight between said first and a second value, yet another percentage having a weight between said second and a third value, and a final percentage having a weight above said third value. Said first, second and third values may be chosen based on what is known about, e.g., biological properties of certain molecular weight fractions of the polysaccharide in question. Such a subdivision of the sample may of course be carried out to the desired degree of resolution, employing the requisite amount of fractions.

A given elution time thus corresponds to a certain molecular weight. To enable analysis of the chromatogram obtained in step (iii) of the method in order to establish the molecular weight distribution of the sample, a set of standard samples of the polysaccharide, each having a known average molecular weight, are preferably analyzed, under the conditions used for the analysis of test samples. This will enable the definition of specific elution times for specific molecular weights. Step (iv) of the method then preferably comprises splitting of the peak or peaks of the chromatogram obtained in step (iii) at the times corresponding to the molecular weight standards. This approach is put into practice in the examples described below. Advantageously, the processing of the obtained chromatogram in order to establish the desired information about molecular weight distribution in the polysaccharide sample is carried out using computer software. Software for computerized processing of chromatograms is commercially available, and furthermore, algorithms for peak integration and peak splitting are known to the person of skill in the art.

In its second aspect, the invention concerns a novel use of anion-exchange chromatography. Thus, it has unexpectedly been found that such chromatography may advantageously be put to use in the analysis of molecular weight distribution in samples of anionic polysaccharides of molecular weight higher than 0.05 MDa. In order to use anion-exchange chromatography for this purpose, no particular modifications of standard equipment or columns need be made. The use of anion-exchange rather than size exclusion chromatography makes possible the analysis of polysaccharides that have previously not been possible to analyze chromatographically, due to too high a molecular weight.

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

The invention will now be further illustrated through the description of examples of its practice. The examples are not intended as limiting in any way of the scope of the invention.

### EXAMPLES

### Example 1

### Determination of the Molecular Weight Distribution of Hyaluronic Acid, 0.1-1 MDa

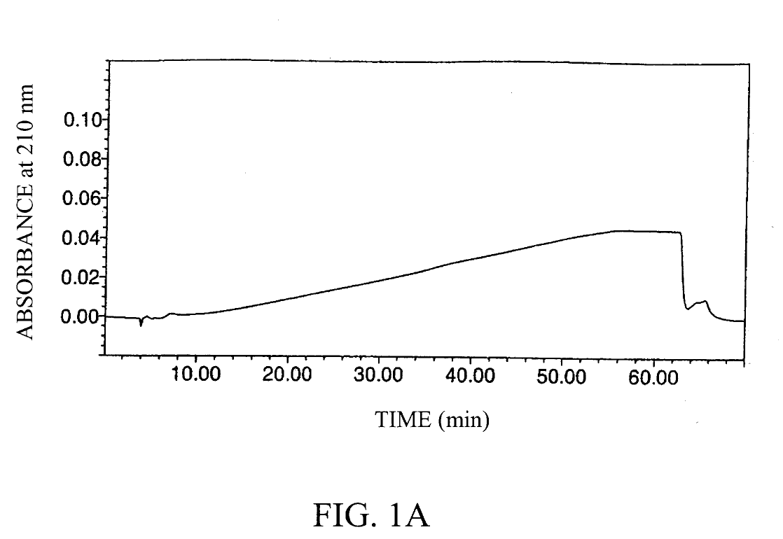

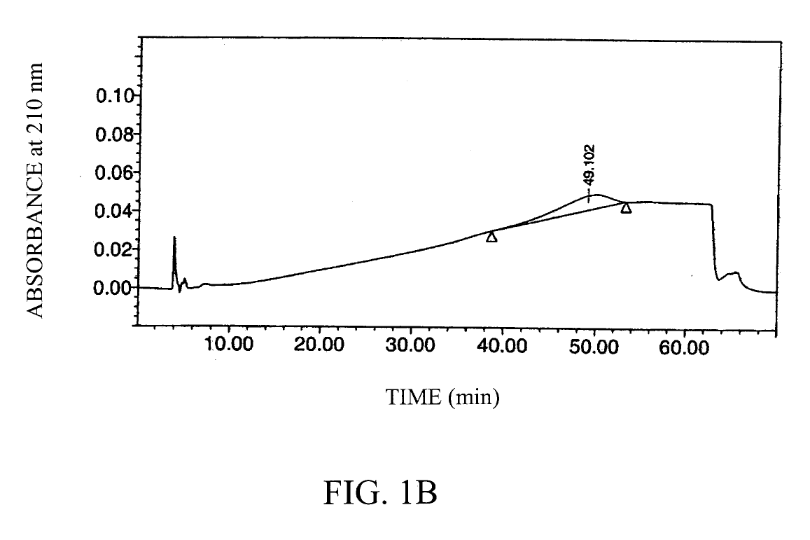

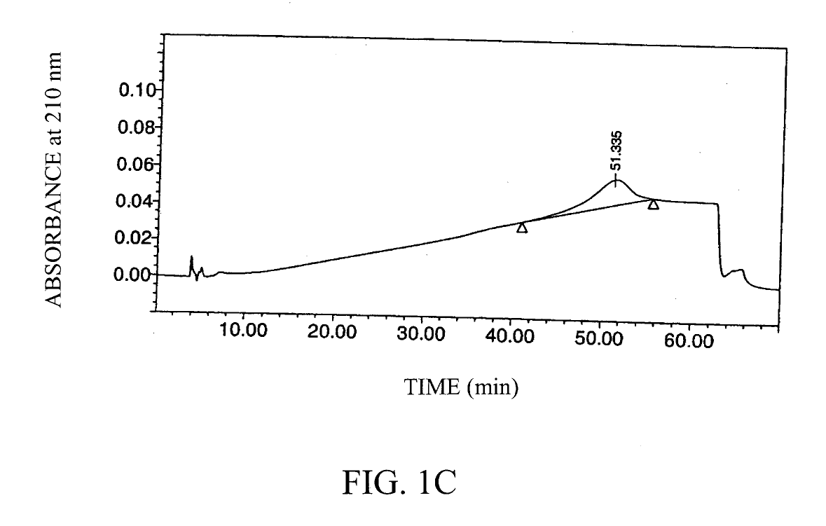

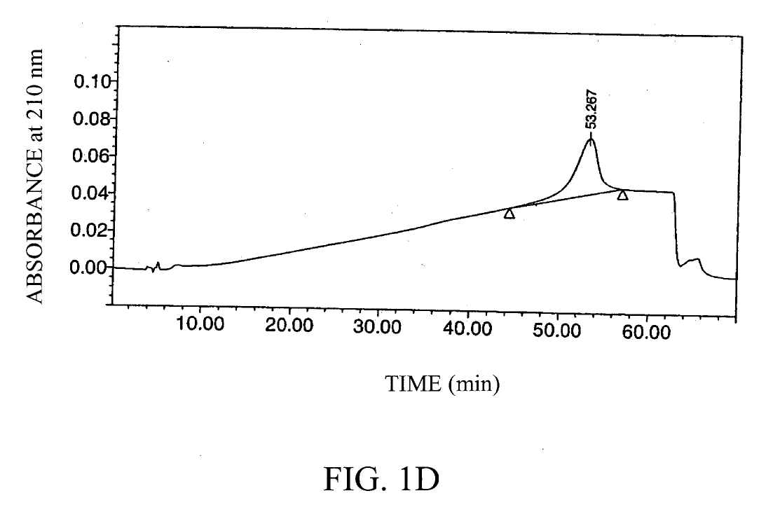

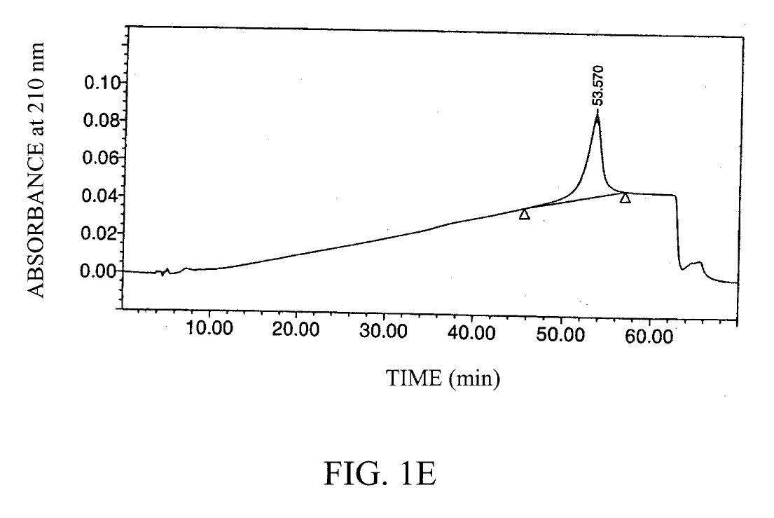

Four laboratory standard samples of hyaluronic acid obtained from Pharmacia AB, Uppsala, Sweden, were analyzed, along with a negative control (water blank; FIG. 1A).

The concentration and average molecular weight of the hyaluronic acid standards were determined by the carbazole method (T Bitter and H M Muir, Anal Biochem 4:330-334 (1962)) and by LALLS, respectively. Average molecular weights of the samples were 0.1, 0.25, 0.5 and 1 MDa. The hyaluronic acid standards were of high purity (>97%), and were diluted to 1 mg/ml in 10 mM Tris/HCl, 20 mM sodium sulfate, pH 8.0.

The hyaluronic acid samples were chromatographed using an HPLC system equipped with a strong anionic-exchange chromatography column, PL-SAX-4000 (4000 A, 8 μm, 150×4.6 mm I.D.), and a PL-SAX precolumn, both purchased from Polymer Laboratories, Ltd (Church Stretton, UK). The functional group of the polymer column is a quaternary amine group.

The column, heated in an oven to 45° C., was equilibrated with 10 mM sodium phosphate, 20 mM sodium sulfate, pH 7.0 (mobile phase A). 10 mM sodium phosphate, 225 mM sodium sulfate, pH 7.0, (mobile phase B) was used for elution. Duplicate injections of 15 μl of each hyaluronic acid standard at a flow rate of 0.5 ml/min were made. Elution was carried out employing a linear gradient from 0 up to 100% of mobile phase B (during 0-50 min), which was followed by a 10 min isocratic run at 100% B, and finally an equilibration time of 10 min in mobile phase A. During the equilibration time, an increased flow of 1 ml/min was used at the time from 60 to 69 min after starting the run. Detection was carried out by measurement of absorbance at 210 nm.

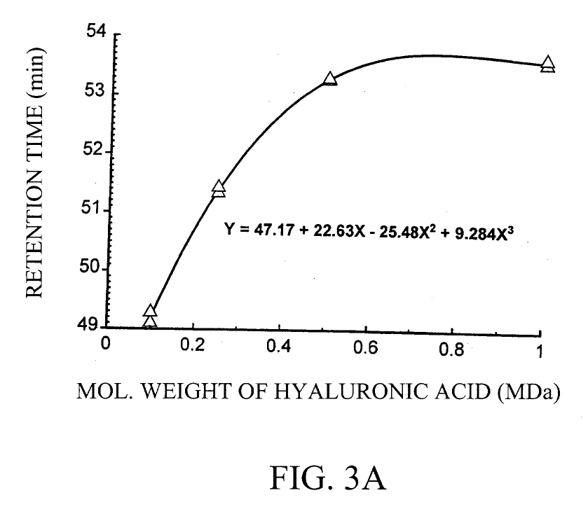

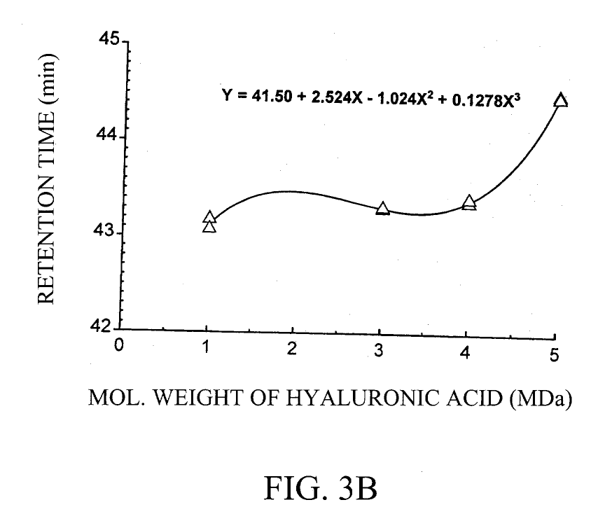

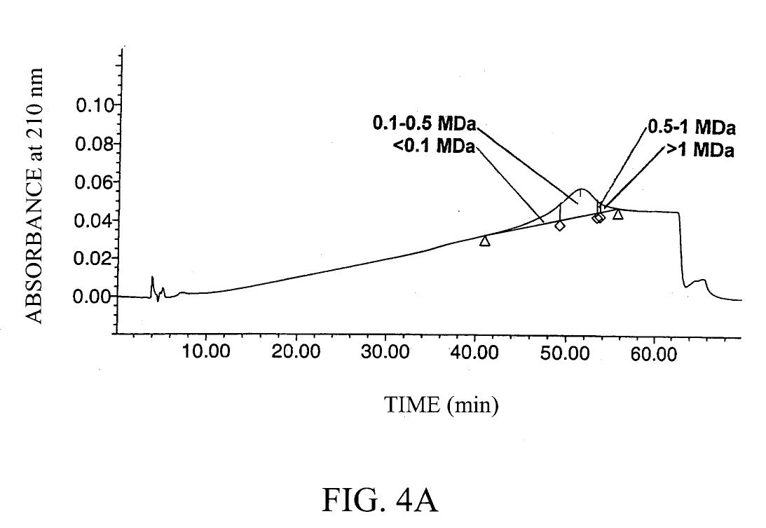

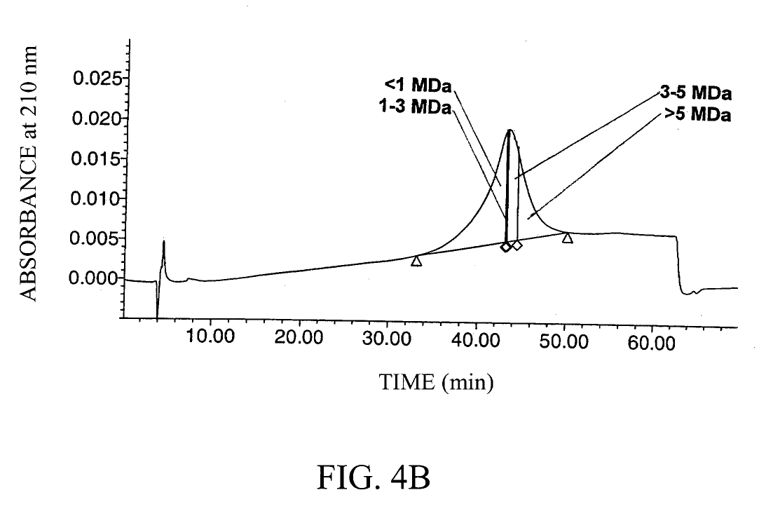

The molecular weight standards of hyaluronic acid with molecular weights of 0.1-1 MDa were eluted with retention times of 49-54 min (FIGS. 1B-1E). A 0.25 MDa hyaluronic acid standard sample, analyzed as an unknown sample in the end of the sequence, had a retention time of 51.40 min (FIG. 4A), which gave a peak molecular weight of 0.25 MDa, calculated from the formula in FIG. 3A.

By manual splitting of the peak at the retention times for the respective molecular weight standard, as shown in FIG. 4A, the following molecular weight distribution was obtained:

| | | |
|---|---|---|
| <0.1 | MDa: | 27% |
| 0.1-0.5 | MDa: | 64% |
| 0.5-1 | MDa: | 2% |
| >1 | MDa: | 6% |

### Example 2

### Determination of the Molecular Weight Distribution of Hyaluronic Acid, 1-5 MDa

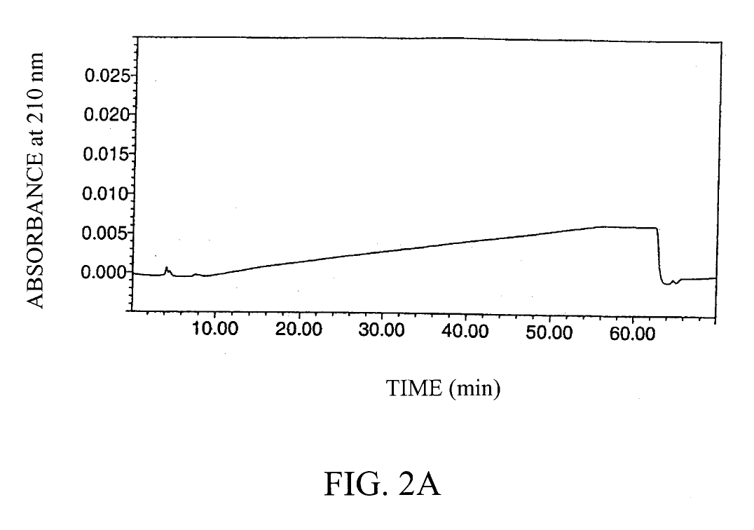

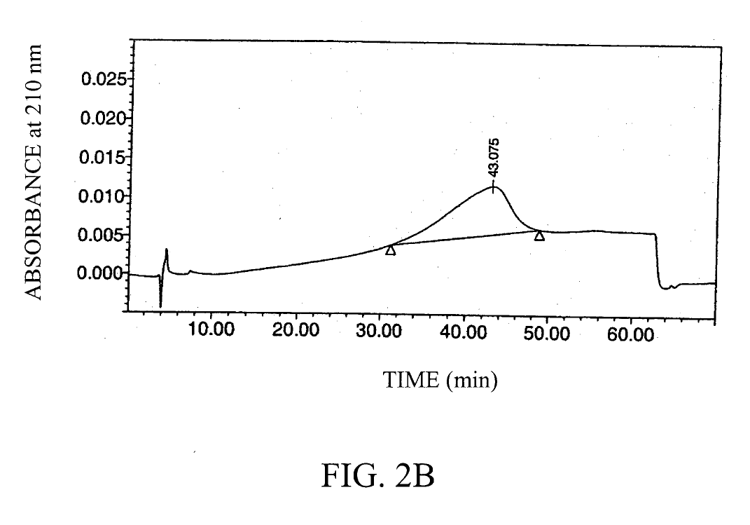

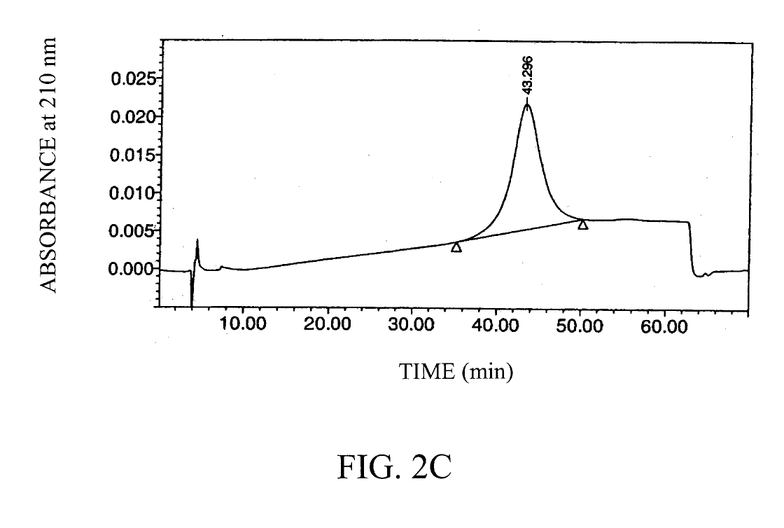

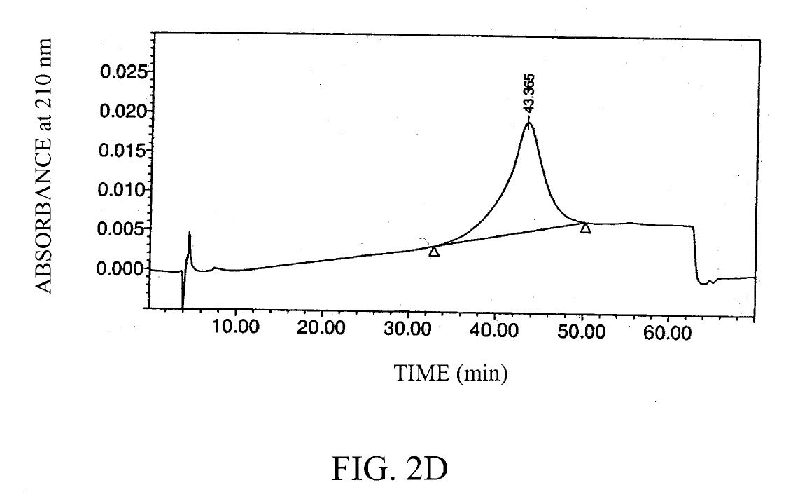

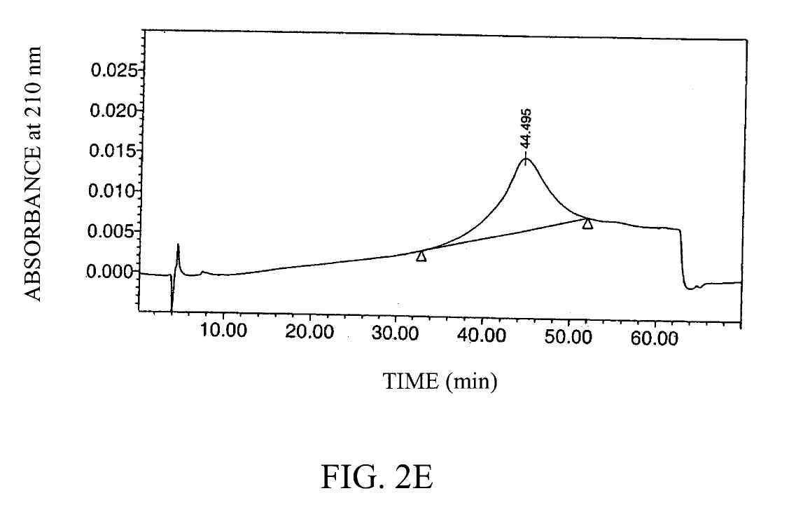

The same equipment and method as in Example 1 was used, except that the hyaluronic acid standard samples used for the standard curve had molecular weights of 1, 3, 4 and 5 MDa, and that the mobile phase A contained 10 mM sodium phosphate, 175 mM sodium sulfate, pH 7.0. Again, a negative control water blank was also analyzed (FIG. 2A).

The molecular weight standards of hyaluronic acid, 1-5 MDa, were eluted with retention times of 43-45 min (FIGS. 2B-2E). A 4 MDa HA standard sample, analyzed as an unknown sample in the end of the sequence, had a retention time of 43.41 min (FIG. 4B), which gave a peak molecular weight of 4.0 MDa, calculated from the formula in FIG. 3B.

By manual splitting of the peak at the retention times for the respective molecular weight standard, as shown in FIG. 4B, the following molecular weight distribution was obtained:

| | | |
|---|---|---|
| <1 | MDa: | 51% |
| 1-3 | MDa: | 3% |
| 3-5 | MDa: | 22% |
| >5 | MDa: | 24% |

Through slightly modifying a suitable software program, e.g., of the type used for molecular weight determination in connection with size exclusion chromatography, it is possible to automatically calculate a molecular weight distribution curve, including details of percentages of molecular forms of polysaccharide in the unknown sample at specified ranges. The results described above were achieved by manual integration using conventional HPLC software. Other evaluation models, e.g., plotting of the weighted average molecular weight for respective peak against the retention time, may also be useful. Herein is used a third degree polynomial curve fitting model for the standard curve, which did not give an optimal fitting. However, for the determination of the actual samples of about 0.25 (Example 1) and 4 MDa (Example 2), the model was considered to be satisfactory.

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

What is claimed is:

1. A method for determining the molecular weight distribution of an anionic polysaccharide in a sample, the method comprising:
(i) providing a sample comprising an anionic polysaccharide with an average molecular weight in the range of 0.05 to 10 MDa;
(ii) applying the sample to an anion-exchange chromatography column so as to immobilize the polysaccharide to the column;
(iii) eluting the immobilized polysaccharide from the column and recording a chromatogram of the amount of polysaccharide eluted as a function of time; and
(iv) determining the molecular weight distribution of the polysaccharide in the sample through analysis of the chromatogram.

2. The method of claim 1, further comprising establishing chromatographic retention times, under the chromatographic conditions used in steps (i)-(iii), for a set of standard samples of the anionic polysaccharide, each of the standard samples having a known mean molecular weight of the anionic polysaccharide, wherein the determination of the molecular weight distribution in step (iv) comprises splitting of peaks of the chromatogram obtained in step (iii) at the retention times for the standard samples.

3. The method of claim 1, wherein the average molecular weight of the anionic polysaccharide in the sample is in the range from 0.1 to 10 MDa.

4. The method of claim 2, wherein the average molecular weight of the anionic polysaccharide in the sample is in the range from 0.1 to 10 MDa.

5. The method of claim 3, wherein the average molecular weight of the anionic polysaccharide in the sample is in the range from 0.5 to 5 MDa.

6. The method of claim 1, wherein the pH value in the mobile phase of the chromatographic system is in the range from pH 4 to pH 11.

7. The method of claim 6, wherein the pH value in the mobile phase of the chromatographic system is in the range from pH 6 to pH 9.

8. The method of claim 1, wherein the sample has a degree of purity such that contaminants other than the anionic polysaccharide constitute less than 5% of the negatively charged species in the sample.

9. The method of claim 2, wherein the sample has a degree of purity such that contaminants other than the anionic polysaccharide constitute less than 5% of the negatively charged species in the sample.

10. The method of claim 8, wherein the sample has a degree of purity such that contaminants other than the anionic polysaccharide constitute less than 1% of the negatively charged species in the sample.

11. The method of claim 10, wherein the sample has a degree of purity such that contaminants other than the anionic polysaccharide constitute less than 0.1% of the negatively charged species in the sample.

12. The method of claim 1, wherein the anionic polysaccharide is chondroitin sulfate, keratan sulfate, dermatan sulfate, heparin sulfate, or heparan sulfate.

13. The method of claim 1, wherein the anionic polysaccharide is hyaluronic acid.

14. The method of claim 2, wherein the anionic polysaccharide is hyaluronic acid.

15. The method of claim 1, wherein the anion-exchange chromatography column comprises a functional group selected from the group consisting of aminoethyl, diethylaminoethyl, dimetylaminoethyl, and polyethyleneimine.

16. The method of claim 1, wherein the anion-exchange chromatography column comprises a functional group selected from the group consisting of trimethylaminomethyl, trimethylaminohydroxypropyl, diethyl-(2-hydroxypropyl)aminoethyl, quaternized polyethyleneimine, triethylaminoethyl, trimethylaminoethyl, and 3-trimethylamino-2-hydroxypropyl.

17. The method of claim 1, wherein the eluting is performed using a concentration gradient of an elution salt solution.

18. The method of claim 17, wherein the elution salt is lithium chloride, sodium chloride, potassium chloride, magnesium chloride, calcium chloride, barium chloride, sodium acetate, lithium perchlorate, magnesium sulfate, potassium phosphate, or potassium sulfate.

19. The method of claim 17, wherein the elution salt is a sulfate.

20. The method of claim 19, wherein the elution salt is sodium sulfate.
